(19) United States
(12) Patent Application Publication
(10) Pub. No.: US 2002/0187132 A1
(43) Pub. Date: Dec. 12, 2002

(54) Cardiac gene transfer

(76) Inventors: Christopher G.A. Mcgregor, Rochester, MN (US); Stephano E. Branzoli, Rochester, MN (US); Timothy O'Brien, Rochester, MN (US)

Correspondence Address: MARK S. ELLINGER, PH.D., Fish & Richardson P.C., P.A., Suite 3300, 60 South Sixth Street, Minneapolis, MN, 55402, US

(21) Appl. No.: 09/846,034

(22) Filed: Apr. 30, 2001

(51) Int. Cl. A61K 48/00; C12N 15/85

(52) U.S. Cl. 424/93.21; 435/455

(57) ABSTRACT

Methods for the selective targeting of a transgene to the media of coronary arteries or the highly efficient transfer of a transgene to the myocardium, based on chemical modifications of a normothermic cardiac perfusion system, are described. The described methods can be used to introduce recombinant genes into donor hearts for the treatment of the complications of heart transplantation, including rejection, infection and cardiac allograft vasculopathy. Also described are perfusion solutions and kits and articles of manufacture.

### BACKGROUND OF THE INVENTION

Heart transplantation is an accepted therapy for selected patients with end-stage heart failure. The one-year survival for heart transplant patients is approximately 80%, with more that 50% of patients alive five years post-transplant. See Hosenpud et al., J. Heart Lung Transplant. 16:691-712 (1997). However, limitations to long-term survival include rejection, infection, side effects of immunosuppression and the development of cardiac allograft vasculopathy (CAV), also known as accelerated transplant atherosclerosis. CAV is the major limitation to long-term survival and has an angiographic incidence rate of 50% at five years after transplant. The etiology and pathogenesis of CAV appears to be multi-faceted. Non-immune mechanisms including early ischemia-induced endothelial cell injury, ischemic reperfusion and cytomegalovirus infection may all contribute. Current treatments for CAV are relatively ineffective and re-transplantation is often necessary. Gene therapy, introducing recombinant genes into donor hearts, may offer a therapeutic intervention that could potentially attenuate this complication of heart transplantation.

Several delivery schemes have been explored for gene transfer to the heart, and a number of vector systems have been used for gene transfer to the transplanted heart. See Ardehali et al., J. Thorac. Cardiovasc. Surg. 109:716-720 (1995), Dalesandro et al., J. Thorac. Cardiovasc. Surg. 111:416-422 (1996) and Sawa et al., Circ 92, II479-11482 (1995). Several groups have studied the use of adenoviral vectors. See Lee et al., J. Thorac. Cardiovasc. Surg. 111, 246-252 (1996), Yap et al., Circ. 94, I-53 (1996) and Pellegrini et al., Transpl. Int. 11, 373-377 (1998). A direct single bolus injection of an adenoviral vector encoding β-galactosidase into the coronary arteries of a donor heart preserved at 4° C. resulted in transgene expression. While inefficient, this method was associated with an even distribution of transgene expression throughout the heart. See Pellegrini et al. In contrast, when the virus was recirculated through the donor heart using a perfusion system at 4° C., a ten-fold increase in gene transfer efficiency was observed, with transgene expression predominantly in the right ventricle and the subepicardial region. See Pellegrini et al., J. Thorac. Cardiovasc. Surg. 119, 493-500 (2000). Gene transfer to the heart has also been reported using a Langendorff perfusion system, allowing gene transfer to occur at physiologic temperatures. See Donahue et al., Proc. Natl. Acad. Sci. USA 94:4664-4668 (1997) and Donahue et al., Gene Therapy 5:630-634 (1998). Rapid, efficient cardiac viral gene transfer was achieved when an adenoviral vector was delivered to intact rabbit hearts by intracoronary perfusion in a Langendorff system at a temperature of 35-37° C. In this experiment, the hearts were not studied intact, but cardiomyocytes isolated after the perfusion indicate nearly 100% of myocytes expressed the reporter gene. See Donahue et al., Proc. Natl. Acad. Sci.USA 94:4664-4668 (1997).

### SUMMARY

This invention relates to methods for delivering a transgene to an organ prior to transplantation. Chemical modifications of a cardiac perfusion system allow either selective targeting of a transgene to the media of coronary arteries or highly efficient myocardial gene transfer. This discovery has applications in gene-therapy based approaches for the treatment of cardiac diseases, including, but not limited to, the treatment of cardiac allograft vasculopathy. This discovery has broad applications in the overall field of gene transfer.

In one aspect, the invention features a method of preferentially delivering an exogenous nucleic acid to medial cells in coronary arteries rather than cardiomyocytes by perfusing a mammalian heart with a cardiac perfusion solution having 0.4-0.6 mM Ca²⁺, 80-120 mM Na¹⁺, an endothelial cell permeability enhancing agent, a physiological buffering agent and the exogenous nucleic acid. The cardiac perfusion solution can be 0.5 mM Ca²⁺, 100 mM Na¹⁺, 1.0×10⁻³ mM histamine as the endothelial cell permeability enhancing agent and 20 mM Hepes as the physiological buffering agent. The cardiac perfusion solution can be at a temperature in the range of 28-39° C., including at a temperature of 37° C. The perfused heart can be ex vivo or in vivo. The exogenous nucleic acid can be a viral vector transgene construct. The method can be used to reduce the risk of cardiac allograft vasculopathy in a cardiac allograft or xenograft by delivering an exogenous nucleic acid to the allograft or xenograft; the exogenous nucleic acid delivered to reduce the risk of such may encode eNOS.

In another aspect, the invention features a method of efficiently delivering an exogenous nucleic acid to cardiomyocytes by perfusing a mammalian heart with a cardiac perfusion solution having 0.04-0.06 mM Ca²⁺, 40-60 mM Na¹⁺, an endothelial cell permeability enhancing agent, a physiological buffering agent and the exogenous nucleic acid. The cardiac perfusion solution can be 0.05 mM Ca²⁺, 50 mM Na¹⁺, 1×10⁻² mM histamine as the endothelial cell permeability enhancing agent and 20 mM Hepes as the physiological buffering agent. The cardiac perfusion solution can be at a temperature in the range of 28-39° C., including 37° C. The perfused heart may be ex vivo or in vivo. The exogenous nucleic acid may be a viral vector transgene construct. The method can be used to reduce the risk of cardiac allograft vasculopathy in a cardiac allograft or xenograft by delivering an exogenous nucleic acid to the allograft or xenograft. The exogenous nucleic acid delivered may encode eNOS.

In another aspect, the invention includes an isolated mammalian heart transfected with an exogenous nucleic acid by either of the two methods above. The transfected mammalian heart can be a human or non-human heart, including a porcine heart, and the transfected heart can include a viral vector transgene construct.

In yet another aspect, the invention features a perfused mammalian heart with a perfusion solution having 0.4-0.6 mM Ca²⁺, 80-120 mM Na¹⁺, an endothelial cell permeability enhancing agent and a physiological buffering agent. The perfusion solution can be 0.5 mM Ca²⁺, 100 mM Na¹⁺, 1.0×10⁻³ mM histamine as the endothelial cell permeability enhancing agent and 20 mM Hepes as the physiological buffering agent. This perfused heart can further include an exogenous nucleic acid, which can be a viral vector transgene construct, and can be human heart or a non-human heart, including a porcine heart.

Another aspect of the invention features a perfused mammalian heart with a perfusion solution having 0.04-0.06 mM Ca²⁺, 40-60 mM Na¹⁺, an endothelial cell permeability enhancing agent and a physiological buffering agent. The perfusion solution can be 0.05 mM Ca²⁺, 50 mM Na¹⁺, 1×10⁻² mM histamine as the endothelial cell permeability enhancing agent and 20 mM Hepes as the physiological buffering agent. This perfused heart can further include an exogenous nucleic acid, which can be a viral vector transgene construct, and can be human heart or a non-human heart, including a porcine heart.

The invention also features selectively transfected mammalian heart in which an exogenous nucleic acid is present in the medial cells of coronary arteries and is substantially absent from the cardiomyocytes. Also included are selectively transfected mammalian hearts in which a first exogenous nucleic acid is present in the medial cells of coronary arteries and is substantially absent from the cardiomyocytes and a second exogenous nucleic acid is present in cardiomyocytes and is substantially absent from the medial cells of coronary arteries. This mammalian heart can be a human heart or a non-human, including a porcine heart.

In another aspect, the invention features a cardiac perfusion solution having 0.4-0.6 mM Ca²⁺, 80-120 mM Na¹⁺, an endothelial cell permeability enhancing agent and a physiological buffering agent. This cardiac perfusion solution can be 0.5 mM Ca²⁺, 100 mM Na¹⁺, 1.0×10⁻³ mM histamine as the endothelial cell permeability enhancing agent and 20 mM Hepes as the physiological buffering agent. The cardiac perfusion can further include an exogenous nucleic acid. This exogenous nucleic acid can be a viral vector transgene construct.

In yet another aspect, the invention features a cardiac perfusion solution having 0.04-0.06 mM Ca²⁺, 40-60 mM Na¹⁺, an endothelial cell permeability enhancing agent and a physiological buffering agent. The cardiac perfusion solution can be 0.05 mM Ca²⁺, 50 mM Na¹⁺, 1.0×10⁻² mM histamine as the endothelial cell permeability enhancing agent and 20 mM Hepes as the physiological buffering agent. The cardiac perfusion can further include an exogenous nucleic acid. This exogenous nucleic acid can be a viral vector transgene construct.

The invention also features an article of manufacture for the preferential delivery of an exogenous nucleic acid into smooth muscle cells of coronary arteries over cardiomyocytes. This article of manufacture includes a cardiac perfusion buffer having 0.4-0.6 mM Ca²⁺, 80-120 mM Na¹⁺, an endothelial cell permeability enhancing agent and a physiological buffering agent. This article of manufacture also includes packaging material, a label or package insert, indicating that the cardiac perfusion buffer can be used for the preferential delivery of an exogenous nucleic acid into smooth muscle cells of coronary arteries over cardiomyocytes. The cardiac perfusion solution can have 0.5 mM Ca²⁺, 100 mM Na¹⁺, 1.0×10⁻³ mM histamine as the endothelial cell permeability enhancing agent and 20 mM Hepes as the physiological buffering agent. The article of manufacture can also include an exogenous nucleic acid.

Another aspect of the invention includes an article of manufacture for the efficient delivery of an exogenous nucleic acid into cardiomyocytes. This article of manufacture includes cardiac perfusion buffer and packaging material. The cardiac perfusion buffer has 0.04-0.06 mM Ca²⁺, 40-60 mM Na¹⁺, an endothelial cell permeability enhancing agent and a physiological buffering agent. The packaging material includes a label or package insert indicating that the cardiac perfusion buffer can be used for the efficient delivery of an exogenous nucleic acid into cardiomyocytes. The cardiac perfusion buffer can have 0.05 mM Ca²⁺, 50 mM Na¹⁺, 1.0×10⁻² mM histamine as the endothelial cell permeability enhancing agent and 20 mM Hepes as the physiological buffering agent. The article of manufacture can also include an exogenous nucleic acid.

Another aspect of the invention features an article of manufacture for the preferential delivery of an exogenous nucleic acid into smooth muscle cells of coronary arteries over cardiomyocytes. This article of manufacture includes an exogenous nucleic acid and packaging material, a label or package insert, indicating that the exogenous nucleic acid is to be used with a cardiac perfusion buffer having 0.4-0.6 mM Ca²⁺, 80-120 mM Na¹⁺, an endothelial cell permeability enhancing agent and a physiological buffering agent. The label or package insert can indicate that the exogenous nucleic acid is to be used with cardiac perfusion buffer having 0.5 mM Ca²⁺, 100 mM Na¹⁺, 1.0×10⁻³ mM histamine as the endothelial cell permeability enhancing agent and 20 mM Hepes as the physiological buffering agent. The exogenous nucleic acid can be a viral vector transgene construct.

In another aspect, the invention features an article of manufacture for the efficient delivery of an exogenous nucleic acid into cardiomyocytes. This article of manufacture includes an exogenous nucleic acid and packaging material. The packaging material is a label or package insert and indicates that the exogenous nucleic acid is to be used with a cardiac perfusion buffer having 0.04-0.06 mM Ca²⁺, 40-60 mM Na¹⁺, an endothelial cell permeability enhancing agent and a physiological buffering agent. The label or package can indicate the exogenous nucleic acid is to be used with a cardiac perfusion buffer having 0.05 mM Ca²⁺, 50 mM Na¹⁺, 1.0×10⁻² mM histamine as the endothelial cell permeability enhancing agent and 20 mM Hepes as the physiological buffering agent. The exogenous nucleic acid can be a viral vector transgene construct.

In another aspect, the invention features a kit for the preferential delivery of an exogenous nucleic acid into smooth muscle cells of coronary arteries over cardiomyocytes. The kit includes cardiac perfusion buffer and packaging material. The cardiac perfusion buffer has 0.4-0.6 mM Ca²⁺, 80-120 mM Na¹⁺, an endothelial cell permeability enhancing agent and a physiological buffering agent. The packaging material includes a label or package insert indicating that the cardiac perfusion buffer can be used for the preferential delivery of an exogenous nucleic acid into smooth muscle cells of coronary arteries over cardiomyocytes. The cardiac perfusion buffer can be 0.5 mM Ca²⁺, 100 mM Na¹⁺, 1.0×10⁻³ mM histamine as the endothelial cell permeability enhancing agent and 20 mM Hepes as the physiological buffering agent.

In yet another aspect, the invention features a kit for the efficient delivery of an exogenous nucleic acid into cardiomyocytes. The kit includes a cardiac perfusion buffer and packaging material. The cardiac perfusion buffer has 0.04-0.06 mM Ca²⁺, 40-60 mM Na¹⁺, an endothelial cell permeability enhancing agent and a physiological buffering agent. The packaging material includes a label or package insert indicating that the cardiac perfusion buffer can be used for the efficient delivery of an exogenous nucleic acid into cardiomyocytes. The cardiac perfusion buffer can be 0.05 mM Ca²⁺, 50 mM Na¹⁺, 1.0×10⁻² mM histamine as the endothelial cell permeability enhancing agent and 20 mM Hepes as the physiological buffering agent.

Unless otherwise defined, all technical and scientific terms used herein have the same meaning as commonly understood by one of ordinary skill in the art to which the invention belongs. Although methods and materials similar or equivalent to those described herein can be used in the practice and testing of the present invention, suitable methods and materials are described. All publications, patent applications, patents and other references mentioned herein are incorporated by reference in their entirety. In case of conflict, the present specification, including definitions, will control. In addition, the materials, methods, and examples are illustrative only and are not intended to be limiting.

The details of one or more embodiments of the invention are set forth in the accompanying drawings and the description below. Other features, objects, and advantages of the invention will be apparent from the description and drawings, and from the claims

### DESCRIPTION OF DRAWINGS

FIG. 1 depicts light photomicrographs of sections of transplanted hearts transduced with the Ad-β-Gal vector using modified Krebs solution A (0.5 mM Ca⁺², 100 mM Na⁺¹, 20 mM Hepes and 10⁻⁶ M histamine) as the perfusion solution. Transgene expression is predominantly in blood vessels (top 2 panels 40× magnification, lower left 10× magnification, lower right 20× magnification). The panel on the top left is stained with elastic Van Giesen to show the position of the internal elastic lamina. The blue-stained cells indicate β-galactosidase transgene expression.

FIG. 2 depicts a light photomicrograph of a section of a transplanted heart transduced with the Ad-β-Gal vector using modified Krebs solution B (0.05 mM Ca⁺², 50 mM Na⁺¹, 20 mM Hepes and 10⁻⁵ M histamine) as the perfusion solution. Transgene expression is predominantly in cardiomyocytes (1× magnification). The blue-stained cells indicate β-galactosidase expression.

FIG. 3 is a bar graph depicting the NOS activity of AdLacZ and AdeNOS transduced hearts. Determinations were made by measuring the conversion of L-[³H]-citrulline, expressed as mean ±SEM in ρ moles/mg protein/hr (n=6 in each group).

FIG. 4 is a drawing of the apparatus used for the warm continuous perfusion of a donor heart.

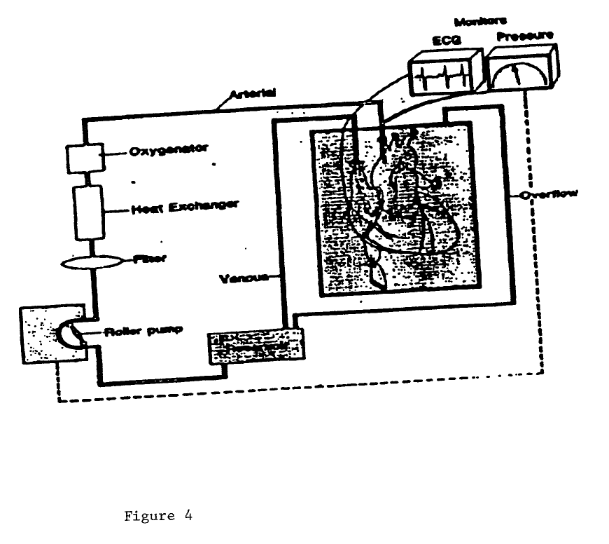

### DETAILED DESCRIPTION

It has been discovered that alterations of the chemical composition of the perfusate in a cardiac perfusion system result in two unambiguous patterns of transgene distribution. In one pattern, gene transfer occurs characteristically in the media of the coronary arteries. In a second pattern, gene transfer is highly efficient and entirely myocardial. For the first time, efficient targeted gene transfer to the coronary arteries has been achieved by chemical modification of a normothermic cardiac perfusion system. Highly efficient myocardial targeting was also achieved. Introducing recombinant genes into donor hearts offers a therapeutic intervention that could potentially attenuate the complications of heart transplantation, including rejection, infection and CAV.

Application of the present discovery, that chemical modifications of the perfusion solution allow either selective targeting of a transgene to the medial cells in the artery or highly efficient gene transfer, is not limited to the field of heart transplantation. The modified perfusion solutions of the present invention may be used to deliver a transgene to coronary arteries as an adjunct to cardiac bypass surgery or during catheter-based coronary intervention procedures. The modified perfusion solutions of the present invention may also be used to deliver a transgene to a saphenous vein grafts. Likewise, application of the present invention is not limited to the treatment of cardiovascular conditions. The modified perfusion solutions may also be used to efficiently target a transgene to the vasculature and other anatomical sites of other tissues and organs, for example, to the afferent or efferent arterioles of the kidney or to the liver or lung. Application of the present invention is not limited to the delivery of an exogenous nucleic acid. Modified perfusion solutions may also be used for the delivery of a therapeutic agent such as a polypeptide, peptide, small organic molecule, peptidomimetic, sugar or lipid. Such polypeptide or peptide agents can comprise naturally-occurring amino acids (e.g., L-amino acids), non naturally occurring amino acids (e.g., D-amino acids) and can be in a linear or cyclic conformation. Peptidomimetics include small molecules that biologically mimic the activity of a polypeptide or a peptide. See Saragovi et al., BioTechnology, 10:773-778 (1992). Therapeutic agents to be delivered may include, but are not limited to, one or more anti-angiogenic agents, such as angiostatin, endostatin, AGM-1470, or TNP-470, alone or in combination with one or more immunosuppressive agents, such as cyclosporine, FK506, steroids, or antiproliferative agents (e.g., azathioprine, mycophenolate moefitil).

Any method and material know to those of skill in the art for the instillation or perfusion of a solution onto a tissue or into an organ can be used to perfuse organs and tissues with the perfusion solutions of the present invention. This includes, for example, but is not limited to, Langendorff perfusion systems, cardiac bypass procedures and catheter-based procedures.

PERFUSION BUFFERS

In the present invention, two different perfusion systems resulted in two unambiguous patterns of transgene distribution. In one pattern, with the use of a perfusion solution comprising 0.4-0.6 mM Ca²⁺, 80-120 mM Na¹⁺, an endothelial cell permeability enhancing agent and a physiological buffering agent, gene transfer occurred characteristically in the media of the coronary arteries. In a second pattern, with the use of a perfusion solution comprising 0.04-0.06 mM Ca²⁺, 40-60 mM Na¹⁺, an endothelial cell permeability enhancing agent and a physiological buffering agent, gene transfer was highly efficient and entirely myocardial.

An endothelial cell permeability-enhancing agent is an agent that effectively increases endothelial cell permeability. See Donahue et al., Gene Ther. 5:630-34 (1998); Ehringer et al., J. Cell Physiol. 167:562-69 (1996); van Nieuw et al., Circ. Res. 83:1115-23 (1998) and Logeart et al., Hum. Gen Ther. 11:1015-22 (2000). Such an agent may be, but is not limited to, an agent selected form the group consisting of histamine, serotonin, bradykinin and thrombin. Preferably an endothelial cell permeability-enhancing agent is histamine at a concentration of 0.1×10⁻⁶ to 5.0×10⁻⁵ mol/l. For example, the histamine concentration may be, but is not limited to, 0.1×10⁻⁶, 0.5×10⁻⁶, 1.0×10⁻⁶, 1.5×10⁻⁶, 5.0×10⁻⁶, 1.0×10⁻⁵, 1.5×10⁻⁵ or 5.0×10⁻⁵ mol/l. Histamine is known to increase microvascular permeability at the level of the post capillary vessel by endothelial cell contraction and opening of endothelial cell junctions and its action is reversible relatively quickly. See Majno et al., J. Biophs. Biochem Cytol. 11:607-26 (1961); He et al., Am J. Physiol. 273:H747-H755 (1997) and van Hinsbegh, Arterioscler. Thromb. Vasc. Biol. 17:1018-23 (1997).

A physiological buffering agent is added to the perfusion solution to optimize perfusate solution pH and enhance solution stability. Many such physiological buffering agents are well known and in wide use. Any of these well known buffering agents may be used in the present invention. For example, a physiological buffering agent may be, but is not limited to, an agent selected from the group consisting of Pipes, Mops, Tes, Hepes, Trizma, Tea and Taps. (Sigma Chemical Co., St. Louis, Mo.). Preferably such a physiological buffering agent is 10-25 mM Hepes, more preferably 20 mM Hepes.

A normothermic perfusion solution may be used in the described perfusion system. In a normothermic perfusion system the perfusion solution is at a temperature approaching physiological temperature. For example, a normothermic perfusion solution may be, but is not limited to, a temperature in the range of 28-39° C. For example, the temperature of a normothermic perfusion solution may be 28, 29, 30, 31, 32, 33, 34, 35, 36, 37, 38 or 39° C.

Perfusion solutions may be sterile. Perfusion solutions may be determined to be pyrogen-free. Methods for determining if a solution is pyrogen-free are well known. See, for example, U.S. Pat. Nos. 5,591,628 and 6,171,807. Perfusion solutions may also be prepackaged in disposable containers for convenient use.

DONOR ORGANS

Transplantation is an ideal setting for gene therapy as the donor organ is available for genetic modification between the time of procurement and implantation. A foreign gene can be transferred to a harvested organ, creating a “transgenic” allograft or xenograft. The current invention features methods of efficiently targeting a foreign gene within a donor organ, based on altering the chemical composition of the perfusate used in a normothermic perfusion system. Targets for the gene transfer methods of the current invention can include any organ, for example heart, lung, liver and kidney. Targets can also include tissues, glands and isolated cell populations. For example, saphenous vein grafts can serve as targets.

Donor organs may be from any mammalian species, including, but not limited to, human, non-human primates such as baboons, monkeys and chimpanzees, miniature swine (porcine), goats, sheep, cows, horses and rabbits and rodents such as rats, guinea pigs and mice. As used herein “organ” and “heart” refer to an organ or heart present in a subject or to an organ or heart that is maintained outside a subject.

VECTORS

Any of a number of different types of vectors is suitable for use in the methods of the invention. For example, plasmid vectors and viral vectors, including but not limited to retroviral vectors, are useful for carrying and delivering the genetic information necessary for the methods of the invention. A large number of plasmids are known to those skilled in the art. The basic requirements of a plasmid vector useful according to the invention are as follows. Useful mammalian plasmid expression vectors will comprise an origin of replication, a suitable promoter and optional enhancer, and also any necessary ribosome binding sites, a polyadenylation site, splice donor and acceptor sites, transcriptional termination sequences, and 5′ flanking nontranscribed sequences. In addition, the expression vectors preferably contain a gene to provide a phenotypic trait for selection of transformed host cells such as dihydrofolate reductase or neomycin resistance for eukaryotic cell culture, or such as tetracycline or ampicillin resistance in E. coli.

Retroviral vectors, which typically transduce only dividing cells, can be used. Adenoviral vectors, capable of delivering DNA to quiescent cells, are currently the agents of choice for cardiovascular gene transfer. See, for example, Ye et al., J. Biol. Chem. 271:3639-3646 (1996). These vectors can be easily manipulated and grown to high titer. First generation adenoviral vectors have a deletion of the EIA region of the adenoviral genome resulting in an inability to replicate. Newer generation adenoviral vectors, which have a deletion of most of the adenoviral sequences, may have less toxicity and a longer duration of transgene expression. See Davis et al., Methods Mol. Biol. 135:515-23 (2000). Another viral vector system with potential advantages is an adeno-associated viral vector. See Clark et al., Hum. Gene Ther., 10(6):1031-1039 (1999) and Liu et al., Gene Therapy, 6:293-299 (1999). This vector system has shown particular promise in skeletal muscle, hepatic and cerebral gene transfer, resulting in prolonged transgene expression without inflammation in these organ systems. Another useful vector system is based on lentiviruses. See Buchschacher and Wong-Staal, Blood 95:2499-2504 (2000); Naldini and Verma, Adv. Virus Res. 55:599 (2000); Vigna and Naldini, J. Gene Med. 2(5):308-16 (2000) and Naldini et al., Science 272:263 (1996). These retroviral vectors are capable of transducing quiescent cells and of integrating DNA into the host cell chromosome.

EXOGENOUS NUCLEIC ACIDS

As used herein, the term “exogenous nucleic acid” refers to a nucleic acid construct, generated by recombinant DNA methods, which is capable of being introduced into a cell, whereupon such construct directs the expression of one or more heterologous gene products within that cell. An exogenous nucleic acid comprises a sequence encoding one or more heterologous gene products and operably linked regulatory elements sufficient to direct the transcription of the sequence encoding the heterologous gene products. An exogenous nucleic acid may also comprise plasmid or viral vector sequences. As used herein, the term “operably linked” means that the two sequences are joined such that the regulatory element is placed in a position and orientation such that expression of the joined coding sequence occurs under the direction of that regulatory element.

TRANSFER eNOS FOR THE TREATMENT OF CAV

Nitrogen monoxide (NO) is formed in mammalian cells from the amino acid L-arginine through the mediation of the enzyme NO synthase (NOS). NO is an important messenger substance and/or signal molecule in the human body that mediates a multitude of physiological and pathophysiological effects. Nitric oxide is an arterial vasodilator that also inhibits proliferation of vascular smooth muscle cells and platelet aggregation. In the cardiovascular system, reduced bioactivity of endothelial nitric monoxide (eNOS) is a feature of atherosclerosis and vascular injury. Using the perfusion system of the present invention to transfer an exogenous nucleic acid encoding eNOS to the heart will be a useful therapeutic intervention for the treatment of cardiovascular disease and as a therapy for cardiac allograft vasculopathy (CAV).

SELECTIVELY TRANSFECTED HEARTS

The methods of the present invention can be used to prepare selectively transfected mammalian hearts. Such a selectively transfected heart is a heart in which an exogenous nucleic acid has been preferentially delivered to some tissues and not to other tissues. For example, a selectively transfected heart may be a heart in which an exogenous nucleic acid has been preferentially delivered to medial cells in the coronary artery and substantially not present above background levels in cardiomyocytes. Such a heart may be prepared by perfusion with a cardiac perfusion solution having 0.4-0.6 mM Ca²⁺, 80-120 mM Na¹⁺, an endothelial cell permeability enhancing agent, a physiological buffering agent and the exogenous nucleic acid. More preferably, a cardiac perfusion solution having 0.5 mM Ca²⁺, 100 mM Na¹⁺, wherein said endothelial cell permeability enhancing agent is 1.0×10⁻³ mM histamine and said physiological buffering agent is 20 mM Hepes and the exogenous nucleic acid may be used.

A selectively transfected heart may be a heart in which an exogenous nucleic acid has been preferentially delivered to cardiomyocytes. Such a heart may be prepared by perfusion with a cardiac perfusion solution having 0.04-0.06 mM Ca²⁺, 40-60 mM Na¹⁺, an endothelial cell permeability-enhancing agent, a physiological buffering agent and the exogenous nucleic acid. Preferably, a cardiac perfusion solution having 0.05 mM Ca²⁺, 50 mM Na¹⁺, said endothelial cell permeability enhancing factor is 1×10⁻² mM histamine, said physiological buffering agent is 20 mM Hepes and the exogenous nucleic acid may be used.

A selectively transfected heart may also be a heart in which a first exogenous nucleic acid has been preferentially delivered to medial cells in the coronary artery and rather than cardiomyocytes and a second exogenous nucleic acid has been preferentially delivered to cardiomyocytes. Such a heart may be prepared by repeated transfection. The first exogenous nucleic acid may be delivered in a cardiac perfusion solution having 0.4-0.6 mM Ca²⁺, 80-120 mM Na¹⁺, an endothelial cell permeability-enhancing agent and a physiological buffering agent. The second exogenous nucleic acid may be delivered in a cardiac perfusion solution having 0.04-0.06 mM Ca²⁺, 40-60 mM Na¹⁺, an endothelial cell permeability-enhancing agent and a physiological buffering agent. More preferably, a first cardiac perfusion solution, having 0.5 mM Ca²⁺, 100 mM Na¹⁺, said an endothelial cell permeability enhancing agent is 1.0×10⁻³ mM histamine, said physiological buffering agent is 20 mM Hepes and the first exogenous nucleic acid, and a second cardiac perfusion solution, having 0.05 mM Ca²⁺, 50 mM Na¹⁺, wherein said endothelial cell permeability enhancing agent is 1×10⁻² mM histamine, said physiological buffering agent is 20 mM Hepes and the second exogenous nucleic acid, may be used. The first and second exogenous nucleic acids may be delivered to the heart in any order.

KITS AND ARTICLES OF MANUFACTURE

In further embodiments, the present invention includes kits or articles of manufacture for conveniently and effectively carrying out the methods in accordance with the present invention. This includes articles of manufacture for the preferential delivery of an exogenous nucleic acid into smooth muscle cells of coronary arteries over cardiomyocytes, including a cardiac perfusion buffer having 0.4-0.6 mM Ca²⁺, 80-120 mM Na¹⁺, an endothelial cell permeability enhancing agent, a physiological buffering agent and packaging material, wherein the packaging material includes a label or package insert indicating that the cardiac perfusion buffer can be used for the preferential delivery of an exogenous nucleic acid into smooth muscle cells of coronary arteries over cardiomyocytes. The cardiac perfusion solution may have 0.5 mM Ca²⁺, 100 mM Na¹⁺, 1.0×10⁻³ mM histamine as the endothelial cell permeability enhancing agent and 20 mM Hepes as the physiological buffering agent.

This also includes articles of manufacture for the efficient delivery of an exogenous nucleic acid into cardiomyocytes, including a cardiac perfusion buffer having 0.04-0.06 mM Ca²⁺, 40-60 mM Na¹⁺, an endothelial cell permeability enhancing agent, a physiological buffering agent and packaging material, wherein the packaging material includes a label or package insert indicating that the cardiac perfusion buffer can be used for the efficient delivery of an exogenous nucleic acid into cardiomyocytes. The cardiac perfusion solution may have 0.05 mM Ca²⁺, 50 mM Na¹⁺, 1×10⁻² mM histamine as the endothelial cell permeability enhancing agent and 20 mM Hepes as the physiological buffering agent.

Each article of manufacture may further comprise an exogenous nucleic acid and packaging material, the packaging material including a label or package insert indicating that the exogenous nucleic acid is to be used with the cardiac perfusion buffer. The exogenous nucleic acid may further comprise a viral vector transgene construct.

### EXAMPLES

### Example 1

### Perfusion Solutions

Modified Krebs solution A was 0.5 mM Ca²⁺, 100 mM Na¹⁺, 20 mM Hepes and 10⁻⁶ M histamine. Modified Krebs solution B was 0.05 mM Ca⁺², 50 mM Na⁺¹, 20 mM Hepes and 10⁻⁵ M histamine. University of Wisconsin solution (UWS) was as described in Belzer et al., U.S. Pat. No. 4,798,824.

### Example 2

### Adenoviral Vectors

A replication defective E1a deleted serotype 5 adenoviral vector encoding nonnuclear-targeted Escherichia coli β-galactosidase under the control of the cytomegalovirus promoter (Ad-β-Gal) was used in this study. (Provided by James Wilson, Institute for Gene Therapy, University of Pennsylvania”) This vector has been rendered replication defective by replacing the entire E1a and most of the E1b regions of the adenoviral genome with the complementary DNA expression cassette. A similar adenoviral vector without an insert (Adeno-Null) served as a control vector. Recombinant virus was propagated in transformed human embryonic kidney carcinoma cells (“293 cells”), that constitutively express E1 proteins, isolated and purified as described in Graham and Prevec (Manipulation of adenovirus vectors, in Methods in Molecular Biology (ed. Murray, E. J.) pp. 109-128 (The Humana Press, Clifton, 1991)) and stored at −80° C. in a buffered solution of 10% glycerol until use. Viral titers were determined by plaque assay and expressed as plaque forming units per milliliter (pfu/ml).

### Example 3

### Animals

Rats—Inbred Lewis (270-330 grams) and Brown Norway rats were used as donors and recipients for transplants. Procedures and handling of animals were in compliance with “Principles of Laboratory Animal Care” formulated by the National Society for Medical Research, and the “Guide for the Care and Use of Laboratory Animals” prepared by the Institute of Laboratory Animals Resources and published by the National Institute of Health (NIH Publication No. 86-23, revised 1985). All rats were purchased from Harlan Sprague-Dawley, Inc.

### Example 4

### Donor Operation

After anesthesia the donor was intubated and ventilated. A median sternotomy was performed. The cavae, the aorta and the great vessels were isolated. Three hundred units of heparin were injected through the inferior vena cava. The right innominate artery was cannulated with a 24-gauge cannula. Dividing the pulmonary veins and inferior vena cava isolated the heart and the aortic arch was tied distally. At this point, gene transduction was carried out using either a Normothermic Perfusion System or a Hypothermic Perfusion System, as described in sections below.

### Example 5

### Gene Transfer

Five experimental groups were studied (n=6 in Groups 1-4; n=3 in Group 5). With Group 1, the Ad-β-Gal viral transgene (3×10⁹) was delivered to the heart in modified Krebs solution A (0.5 mM Ca⁺², 100 mM Na⁺¹, 20 mM Hepes and 10⁻⁶ M histamine) by Langendorff perfusion for 20 minutes at 37° C. Group 2 was identical to Group 1 except that the Adeno-Null viral transgene was delivered. For Group 3, the Ad-β-Gal viral transgene (3×10⁹) was delivered in modified Krebs solution B (0.05 mM Ca+2, 50 mM Na⁺¹, 20 mM Hepes and 10⁻⁵ M histamine). Group 4 was identical to Group 3, except that the Adeno-Null viral transgene was delivered. With Group 5, the Ad-β-Gal viral transgene (3×10⁹) was delivered in University of Wisconsin solution (UWS) at 4° C. All hearts were examined for transgene expression 7 days after transplantation. Gene transfer was carried out using either a normothermic perfusion system or a hypothermic perfusion system, as outlined below.

### Example 6

### Normothermic Perfusion System

For Groups 1-4, normothermic perfusion with modified Krebs solutions A or B with 95% O₂ and 5% CO₂ (pH 7.4 at 37° C.) was begun through the cannula in situ for 10 minutes to flush any remaining red blood cells from the coronary arteries. The beating heart was then excised and placed in a Langendorff apparatus for an additional 20 minutes of perfusion at 37° C. for gene transfer using the previously placed cannula in the innominate artery. In Groups 1 and 4, Ad-β-Gal virus (3×10⁹) was delivered in modified Krebs solutions A or B, respectively. In Groups 2 and 4, Adeno-Null virus (3×10⁹) was delivered instead. During the period of normothermic perfusion with the modified Krebs solutions, the perfusate flow rate was adjusted to maintain a mean aortic pressure of 70-80 mmHg. The perfusate draining from the IVC was collected and used for recirculation for 20 minutes by means of a peristaltic pump (Rainin, Emeryville, Calif.) in order to achieve recirculation of the adenoviral vector through the coronary vasculature. Hearts were then removed from the Langendorff and stored in UW solution at 4° C. for 40 to 50 minutes for myocardial preservation purposes prior to transplant.

### Example 7

### Hypothermic Perfusion System

In Group 5, hypothermic perfusion (4° C.) was achieved as previously described in Pellegrini et al. (J. Thorac. Cardiovasc. Surg., Vol. 119:493-500, March 2000.

### Example 8

### Recipient Operation

Heterotopic heart transplantation was performed in all animals using standard microsurgical techniques, as described in Ono and Lindsey, Improved Technique of Heart Transplantation in Rats. J. Thorac. Cardiovasc. Surg. 57:225-229 (1969). Function of the heart was checked daily by palpation. Seven days after transplantation, the animals were anesthetized with an intraperitoneal injection of pentobarbital sodium (70 mg/kg) and the transplanted heart was removed and flushed with normal saline solution for study.

### Example 9

### Operative Results

All hearts in Groups 1-4 beat with good visual contractility during the total 30 minutes of normothermic perfusion (10 minutes in situ, 20 minutes of Langendorff perfusion) with either of the modified Krebs solutions. All hearts stopped rapidly after being placed in cold UW solution after this period of normothermic perfusion. Hearts in Group 5 stopped beating immediately when initial in situ perfusion with cold UWS was begun. In summary, all hearts were perfused for 30 minutes, followed by a cold ischemic time of 40-50 minutes immersed in UW solution at 4° C. as the recipient was being prepared, followed by 10 to 20 minutes of warm ischemia as the transplants were being performed. All but three hearts showed early spontaneous sinus rhythm at time of reperfusion after transplantation, these three, soon thereafter. All hearts showed good contractility at the time of explant, 7 days after transplantation. Operative mortality was 8%.

### Example 10

### Assessment of Transgene Expression by X-gal Staining

Expression of the β-galactosidase transgene was evaluated by both X-Gal staining of histologic tissue sections and enzyme linked immunosorbent assay (ELISA) analysis of tissue homogenates. For X-Gal staining of histologic tissue sections, a midventricular section of the excised heart was cut, embedded immediately in OCT compound (Miles Laboratories, Elkhart, Ind.) and snap frozen in a liquid nitrogen-cooled 2-methylbutane bath for 15 minutes. For each of Groups 1-5, five midventricular frozen sections (five microns thick, 50 microns apart) were cut and fixed in 1.25% glutaraldehyde for 15 minutes at 4° C. and rinsed twice with phosphate-buffered saline solution (Gibco BRL, Gaithersburg, Md.). Sections were then stained in a solution of 500 micrograms/ml 5-bromo-4-chloro-3indolyl-[beta]-D-galactopyranside (X-Gal; Boehringer Mannheim Corp, Indianapolis, Ind.) for 4 hours at 37° C. and then rinsed in water and counterstained with eosin. Blue staining cells indicate the presence of β-galactosidase expression. In each slide 10 high power fields were scanned. For blood vessel X-Gal staining, gene transfer was expressed as a percentage of vessels staining over the number of vessels present in each section and a mean value determined. For myocardial X-Gal staining, gene transfer was expressed as a percentage of myocardial cells staining over the total number of cells present in the field and a mean value determined.

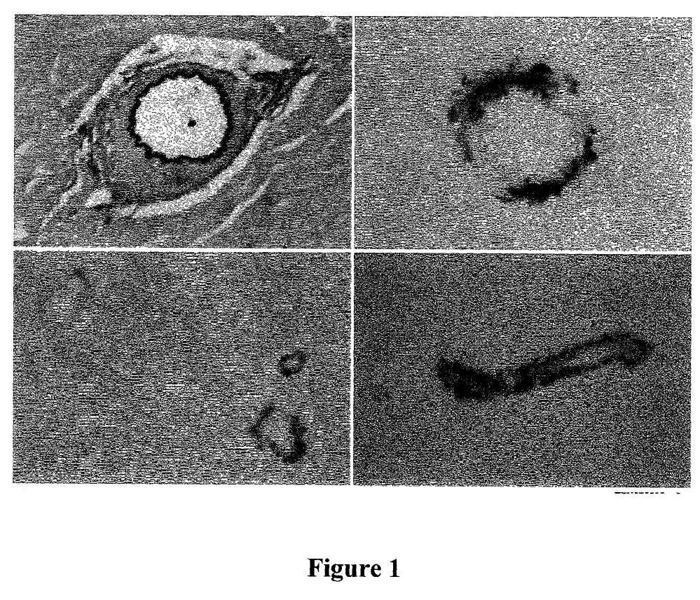

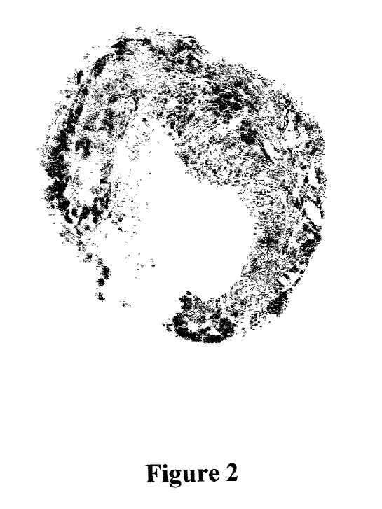

Two clear and unambiguous patterns of transgene expression were evident. In all six animals in Group 1 (Krebs solution A), β-galactosidase expression was present nearly exclusively in smooth muscle cells in the media of the coronary arteries. As shown in FIG. 1, both epicardial and intramyocardial arteries stained blue, indicating β-galactosidase expression. Blue staining cells are seen in the blood vessel wall deep to the internal elastic lamina. The number of positively staining vessels was 10-55% (mean 32%, median 42%). In one heart in this group rare cardiomyocytes (0.6%) expressed the transgene. Group 2 (Adeno-Null) showed no staining for β-galactosidase expression. In Group 3 (Krebs solution B) transduction was highly efficient and entirely myocardial. The number of positively staining cells was 30-95% (mean 62.5%, median 84%). As shown in FIG. 2, only cardiomyocytes are seen to express the transgene. Expression is evident throughout the myocardium in both ventricles. No blue β-galactosidase staining was seen in the Adeno-Null control hearts. There was uniform and widespread myocardial distribution of the gene. No vessels stained. Group 4 (Adeno-Null) showed no staining for β-galactosidase expression. In Group 5 (UW solution) only myocardial gene transfer occurred in 14% to 42% of cells (mean 28%, median 28%).

### Example 11

### Assessment of Transgene Expression by ELISA

Expression of the β-galactosidase transgene was evaluated by both X-Gal staining of histologic tissue sections and enzyme linked immunosorbent assay (ELISA) analysis of tissue homogenates. The ELISA sandwich immunoassay for detecting and measuring the β-galactosidase protein is highly sensitive, with a lower limit of detection of 100 pg of β-galactosidase. Fifty mg of tissue is required for the test. After a section of heart was cut and stored in formalin for histologic assessment, the remaining heart was snap frozen in liquid nitrogen and homogenized (Tekmmar tissue homogenizer, Cincinnati, Ohio) for three minutes in ice-cold buffer (100 mmol/l of potassium phosphate [pH 7.8], 0.2% Triton X-100 [Sigma Chemical Company, St Louis, Mo.] and 200 mmol/l phenylmethylsulfonil fluoride). The homogenate was centrifuged at 18000 g for 10 minutes at 4° C. The supernatant was collected, aliquoted and frozen at −80° C. Transgene expression was quantitatively assessed by means of an enzyme-linked immunosorbent assay (5′ Prime 3′; Prime Inc, Boulder, Colo.). In brief, a rabbit polyclonal antibody specific to the E. coli β-galactosidase protein is coated onto polystyrene microwells. Transgene protein present in tissue extracts is captured and bound to the solid phase. A biotinylated secondary antibody to β-galactosidase then binds to the immobilized primary antibody-β-galactosidase complex. The biotinylated antibody is quantified calorimetrically by incubation with streptavidin-conjugated alkaline phosphatase and color development substrate. Spectrophotometric analysis is performed on an automated analyzer (SPECTRAmax 340; Molecular Devices Corporation, Sunnyvale, Calif.).

The mean β-Gal content for each of Groups 1-5 is shown in Table 1. Statistical comparisons between the groups in β-Gal content are shown in Table 2.

TABLE 1
GROUP β-GALACTOSIDASE CONTENT (ng/mg of protein)

| | MEAN ± SD | MEDIAN |
|---|---|---|
| GROUP 1 | 6.6 ± 7.1 | 4.6 |
| GROUP 2 | 0.2 | 0.5 |
| GROUP 3 | 574 ± 332 | 594 |
| GROUP 4 | 0.3 | 0.2 |
| GROUP 5 | 44.0 ± 34.2 | 56 |

TABLE 2
β-Gal CONTENT-STATISTICAL DATA

| GROUPS | P VALUE |
|---|---|
| 1 vs 2 | <0.01 |
| 1 vs 3 | <0.005 |
| 3 vs 4 | <0.01 |
| 3 vs 5 | <0.05 |

### Example 12

### Histological Assessment of Inflammatory Response

To determine any inflammatory response to the virus or any ischemic injury, formalin fixed sections of heart were cut and stained with hematoxylin and eosin. An experienced pathologist blinded to the origin of the slides graded inflammation and ischemic damage. Inflammation was scored an a scale comparable with the working formulation for cardiac rejection (Billingham et al., J. Heart Transplant. 9:587-593 (1990)), whereas the following scheme was used for ischemic damage: 0, no ischemic damage; 1, less then 5% of the area of the section; 2, between 5% and 20%; 3, between 20 and 40%; 4, more than 40% of the area. There was no evidence of an inflammatory response in any animal. Ischemic damage was grade 1 in all animals.

### Example 13

### Immunohistochemistry Staining for Factor VIII Antigen

Immunostaining for Factor VIII related antigen was performed using a polyclonal antisera to Factor VIII antigen (DAKO, Carpinteria, Calif., 1:1000). Six sections of paraffin embedded tissue from animals in Group 1 were assessed for endothelial integrity using standard immunohistochemical techniques. Staining for Factor VIII showed positive staining of intact endothelium in coronary vessels that stained positive for β-Gal (Group 1).

### Example 14

### Statistical Analysis

A Student's t-test was used to compare myocardial β-Gal staining between Groups 3 and 5. A P value of <0.05 was considered significant. A Wilcoxon Rank-sum test was used to assess the significance of differences in β-galactosidase content between the groups.

### Example 15

### eNOS Transgene

A recombinant adenovirus containing the cDNA encoding bovine eNOS (AdeNOS) was generated as described in Spector et al., “Construction and Isolation of Recombinant Adenovirus with Gene Replacement,” Methods Mol. Genet. 7:31-44 (1995). The bovine cDNA is as described in Sessa et al., J. Biol. Chem. 267:15247-15276 (1992) and GENBANK® Accession Number M95674.

### Example 16

### Transfer of the eNOS Gene to the Transplanted Heart

Experiments were carried out to assess the feasibility of adenoviral-mediated transfer of recombinant endothelial nitric oxide synthase gene (eNOS) to the transplanted rat heart. Adenoviral vectors for bovine eNOS (AdeNOS) or β-galactosidase (AdLacZ, control) were infused into explanted rat hearts as described by Yap et al. (Cardivascular Res. 42:720-727 (1999)). Briefly, donor hearts were excised and transferred to cardioplegic solution at 4° C. Either the eNOS or the LacZ (control) gene at a concentration of 1×10⁹ pfu/ml (total volume 0.350 ml) was infused over 5 seconds into the coronary arteries via the aortic root. The pulmonary artery was clamped during viral infusion and the viral solution was not flushed out at the end of 60 minutes cold storage prior to performing heart transplantation. Transduced donor hearts were heterotopically transplanted into the abdomen of syngeneic recipient rats. After 4 days, the hearts were excised and examined for distribution and function of the recombinant genes. eNOS expression was detected by measurement of NOS enzymatic activity.

### Example 17

### Enzymatic Assay for eNOS Expression

The eNOS enzymatic assay measures the biochemical conversion of L-arginine to L-citrulline by NOS. The assay was performed by methods originally described by Myatt et al. (Placenta 14:373-383,1993) and modified by Miller and Barber (Am. J. Physiol. 271(40):H668-H673, 1996). In brief, tissue homogenates from mid-ventricular sections of transplanted hearts were prepared and eluted through 10-DG desalting columns (Bio-Rad Laboratories, Hercules, Calif., USA). To quantitate NOS activity, duplicate reactions were carried out in the presence of calcium (total activity) and in the absence of calcium plus EGTA (calcium-independent activity) and in the absence of calcium plus EGTA in the presence of N^{G} monomethyl-L-arginine (L-NMMA; non-specific activity). Reaction mixtures of homogenate (150 μl) and cofactor (150 μl) were incubated at 27° C. for 1 hour. Separation of L-[³H]-citrulline was accomplished using affinity columns containing AG 50W-X8 Na^{+ form} 200-400 mesh resin (Bio-Rad Laboratories). Nitric oxide produced by NOS is presumably in a 1:1 molar ration with L-citrulline and, thus, NOS activity is expressed as pmol of [³H]-L-citrulline produced per mg of protein per hour. Calcium-dependent activity equaled total activity minus calcium-independent activity after correcting for non-specific activity.

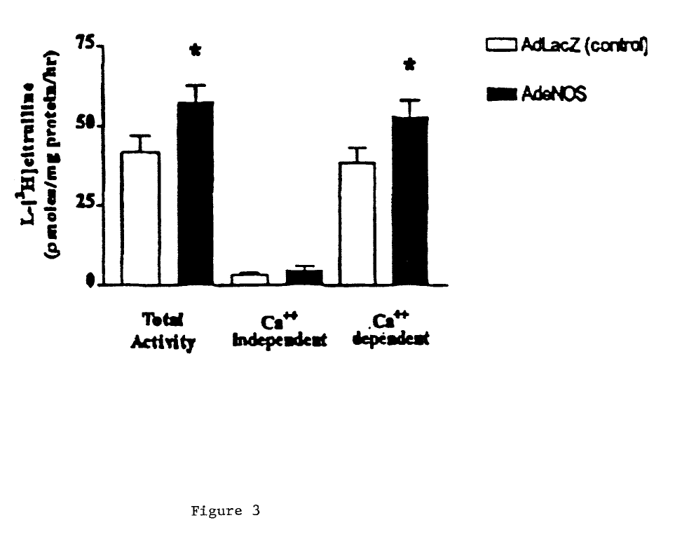

The total NOS activity was 41.7±5.1 pmol L-[³H]-citrulline/mg protein/h in the LacZ-transduced group and 57.7±5.2 pmol/mg protein/h in the eNOS-transduced group (n=6 per group, P=0.05). See FIG. 3. Calcium-dependent activity was 38.4±4.7 pmol/mg protein/h in the LacZ-transduced group vs. 53±5 pmol/mg protein/h in the eNOS-transduced group (P=0.05). Calcium-independent activity of NOS was 3.3±0.5 pmol/mg protein/h in the LacZ-transduced group and 4.7±1.5 pmol/mg protein/h in the eNOS-transduced group (P═NS). Thus, the eNOS transduced hearts showed greater levels of total and calcium-dependent NOS activity compared to LacZ-transduced hearts, P=0.05. Calcium-independent NOS activity was similar in both groups. This study, therefore, demonstrates the feasibility of over expressing eNOS in the transplanted rat heart.

### Example 18

### Adeno-Associated Viral Vectors

To create a recombinant adeno-associated viral (AAV) vector expressing endothelial nitric oxide synthase (eNOS) from the CMV IE promoter-enhancer, the eNOS expression cassette from plasmid pBluescript SK(+) will be excised via XhoI and SmaI digestion and cloned into the AAV plasmid construct pTR-UF5, described in Zolotukhin et al., J. Virol., 70:4646-4654 (1996)). This plasmid contains the humanized GFP reporter gene under the control of a cytomegalovirus immediate-early promoter and a herpes simplex virus thymidine kinase promoter driven neomycin resistance (Neo^{r}) gene cassette inserted between the ITRs of AAV-2. By SalI digestion, blunt ending and subsequent XhoI digestion, the GFP and Neo^{r} gene cassettes will be removed from the pTR-UF 5 construct and replaced by the eNOS expression cassette. The resulting construct, AAV-CMV-eNOS, will be used to generate recombinant viral vector stocks by cotransfection methods as described previously in Li et al., J. Virol. 71(7):5236-5243 (1997); Xiao et al., J. Virol. 72:2224-2232 (1998) and Bartlett et al., J. Virol. 74(6):2777-2785 (2000). Vectors will be purified from clarified cell lysates by non-ionic iodixanol gradient separation and heparan sulfate affinity chromatography as described in Clark et al., Hum. Gene Ther. 10(6):1031-1039 (1999) and Zolotukhin et al., Gene Ther., 6:973-985 (1999). Vector titers range from 2×10¹⁰ to 5×10¹¹viral particles per ml (approximately 10⁷ to 10⁸ IU (infectious units) of virus per ml). Preparations of vector produced by the cotransfection method will be used initially to screen vector constructs for gene expression and efficacy in vitro. To provide adequate virus for animal studies, stable rAAV/CMV-eNOS producer cell lines will be generated. To this end, the eNOS expression cassette from pBluescript SK(+) plasmid will be cloned into the AAV plasmid construct pTPΔNot. For establishment of the producer cell line, a plasmid which contains the AAV genetic elements necessary for wild-type free rAAV production will be constructed. Three domains need be present in this plasmid; (i) the rAAV vector pAAv/CMV-eNOS (AAV terminal repeats flanking the eNOS expression cassette), (ii) AAV helper functions encoded by the AAV Rep and AAV Cap genes, and (iii) a neomycin resistance selectable marker gene. The TPΔNot plasmid contains the later 2 of these requirements and a convenient NotI restriction endonuclease site preceded by the CMV I/E promoter for insertion of the eNOS expression cassette and generation of the third requirement. Construction of similar rAAV tripartite vectors is described Clark et al., Hum. Gene Ther. 6(10):1329-1341 (1995). Subsequent preparations of rAAV/CMV-eNOS will be obtained by infection of producer cell lines with adenovirus (Ad 5). Cell lines will be prepared by transfection of HeLa cells with the tripartite vector described above followed by selection for neomycin resistance. Resulting clones will be characterized and selected for high-level rAAV production as described in Clark et al. (1995) and Liu et al., Gene Ther., 6:293-299 (1999). Purification of rAAV/CMV-eNOS from producer cell lines will be accomplished by heparin affinity chromatography as described in Yamada et al., J. Thorac. Cardiovasc. Surg. 119:709-719 (2000) and Graham et al., J. Gen. Virol., 36(1):1977. Yields of rAAV particles produced from such producer cell lines approach, and often exceed, 10¹⁵ particles.

What is claimed is:

1. A method of preferentially delivering an exogenous nucleic acid to medial cells in coronary arteries rather than cardiomyocytes comprising perfusing a mammalian heart with a cardiac perfusion solution, wherein said cardiac perfusion solution comprises 0.4-0.6 mM Ca²⁺, 80-120 mM Na¹⁺, an endothelial cell permeability enhancing agent, a physiological buffering agent and said exogenous nucleic acid.

2. The method of claim 1, wherein said cardiac perfusion solution comprises 0.5 mM Ca²⁺, 100 mM Na¹⁺, said endothelial cell permeability enhancing agent is 1.0×10⁻³ mM histamine and said physiological buffering agent is 20 mM Hepes.

3. A method of efficiently delivering an exogenous nucleic acid to cardiomyocytes comprising perfusing a mammalian heart with a cardiac perfusion solution, wherein said cardiac perfusion solution comprises 0.04-0.06 mM Ca²⁺, 40-60 mM Na¹⁺, an endothelial cell permeability enhancing agent, a physiological buffering agent and said exogenous nucleic acid.

4. The method of claim 3, wherein said cardiac perfusion solution comprises 0.05 mM Ca²⁺, 50 mM Na¹⁺, said endothelial cell permeability enhancing agent is 1×10⁻² mM histamine and said physiological buffering agent is 20 mM Hepes.

5. The method of either one of claims 1 or 3, wherein said cardiac perfusion solution is at a temperature of 28-39° C.

6. The method of either one of claims 1 or 3, wherein said cardiac perfusion solution is at a temperature of 37° C.

7. The method of either one of claims 1 or 3 wherein said heart is ex vivo.

8. The method of either one of claims 1 or 3 wherein said heart is in vivo.

9. A method of reducing the risk of cardiac allograft vasculopathy in a cardiac allograft or xenograft comprising delivering an exogenous nucleic acid to said cardiac allograft or xenograft by the method of claim 1 or 3.

10. The method of claim 9, wherein said exogenous nucleic acid encodes eNOS.

11. The method of claim 1 or 3, wherein said exogenous nucleic acid comprises a viral vector transgene construct.

12. An isolated mammalian heart transfected with an exogenous nucleic acid by the method of claim 1 or 3.

13. The isolated heart of claim 12, wherein said mammalian heart is a human heart.

14. The isolated heart of claim 12, wherein said mammalian heart is a non-human heart.

15. The isolated heart of claim 12, wherein said mammalian heart is a porcine heart.

16. The isolated heart of claim 12, wherein said exogenous nucleic acid comprises a viral vector transgene construct.

17. A mammalian heart comprising a perfusion solution, said perfusion solution comprising 0.4-0.6 mM Ca²⁺, 80-120 mM Na¹⁺, an endothelial cell permeability enhancing agent and a physiological buffering agent.

18. The mammalian heart of claim 17, wherein said perfusion solution comprises 0.5 mM Ca²⁺, 100 mM Na¹⁺, said endothelial cell permeability enhancing agent is 1.0×10⁻³ mM histamine and said physiological buffering agent is 20 mM Hepes.

19. A mammalian heart comprising a perfusion solution, said perfusion solution comprising 0.04-0.06 mM Ca²⁺, 40-60 mM Na¹⁺, an endothelial cell permeability enhancing agent and a physiological buffering agent.

20. The mammalian heart of claim 19, wherein said perfusion solution comprises 0.05 mM Ca²⁺, 50 mM Na¹⁺, said endothelial cell permeability enhancing agent is 1×10⁻² mM histamine and said physiological buffering agent is 20 mM Hepes.

21. The perfused heart of claim 17 or 19, wherein said perfusion solution further comprises an exogenous nucleic acid.

22. The perfused heart of claim 21, wherein said exogenous nucleic acid comprises a viral vector transgene construct.

23. The perfused heart of claim 17 or 19, wherein said mammalian heart is a human heart.

24. The perfused heart of claim 17 or 19, wherein said mammalian heart is a non-human heart.

25. The perfused heart of claim 17 or 19, wherein said mammalian heart is a porcine heart.

26. A cardiac perfusion solution comprising 0.4-0.6 mM Ca²⁺, 80-120 mM Na¹⁺, an endothelial cell permeability enhancing agent and a physiological buffering agent

27. The cardiac perfusion solution of claim 26, wherein said perfusion solution comprises 0.5 mM Ca²⁺, 100 mM Na¹⁺, said endothelial cell permeability enhancing agent is 1.0×10⁻³ mM histamine and said a physiological buffering agent is 20 mM Hepes.

28. A cardiac perfusion solution comprising 0.04-0.06 mM Ca²⁺, 40-60 mM Na¹⁺, an endothelial cell permeability enhancing agent and a physiological buffering agent.

29. The cardiac perfusion solution of claim 28, wherein said cardiac perfusion solution comprises 0.05 mM Ca²⁺, 50 mM Na¹⁺, said endothelial cell permeability enhancing agent is 1.0×10⁻² mM histamine and said physiological buffering agent is 20 mM Hepes.

30. The cardiac perfusion solution of claim 26 or 28, further comprising an exogenous nucleic acid.

31. The cardiac perfusion solution of claim 30 wherein said exogenous nucleic acid comprises a viral vector transgene construct.

32. An article of manufacture for the preferential delivery of an exogenous nucleic acid into smooth muscle cells of coronary arteries over cardiomyocytes, said article of manufacture comprising cardiac perfusion buffer and packaging material, wherein said cardiac perfusion buffer comprises 0.4-0.6 mM Ca²⁺, 80-120 mM Na¹⁺, an endothelial cell permeability enhancing agent and a physiological buffering agent, wherein said packaging material comprises a label or package insert indicating that said cardiac perfusion buffer can be used for the preferential delivery of an exogenous nucleic acid into smooth muscle cells of coronary arteries over cardiomyocytes.

33. The article of manufacture of claim 32, wherein said cardiac perfusion buffer comprises 0.5 mM Ca²⁺, 100 mM Na¹⁺, said an endothelial cell permeability enhancing agent is 1.0×10⁻³ mM histamine and said a physiological buffering agent is 20 mM Hepes.

34. An article of manufacture for the efficient delivery of an exogenous nucleic acid into cardiomyocytes, said article of manufacture comprising cardiac perfusion buffer and packaging material, wherein said cardiac perfusion buffer comprises 0.04-0.06 mM Ca²⁺, 40-60 mM Na¹⁺, an endothelial cell permeability enhancing agent and a physiological buffering agent, wherein packaging material comprises a label or package insert indicating that said cardiac perfusion buffer can be used for the efficient delivery of an exogenous nucleic acid into cardiomyocytes.

35. The article of manufacture of claim 34, wherein said cardiac perfusion buffer comprises 0.05 mM Ca²⁺, 50 mM Na¹⁺, said endothelial cell permeability enhancing agent is 1.0×10⁻² mM histamine and said physiological buffering agent is 20 mM Hepes.

36. The article of manufacture of claim 32 or 34 further comprising an exogenous nucleic acid.

37. An article of manufacture for the preferential delivery of an exogenous nucleic acid into smooth muscle cells of coronary arteries over cardiomyocytes, said article of manufacture comprising an exogenous nucleic acid and packaging material, wherein said packaging material comprises a label or package insert indicating that said exogenous nucleic acid is to be used with cardiac perfusion buffer comprising 0.4-0.6 mM Ca²⁺, 80-120 mM Na¹⁺, an endothelial cell permeability enhancing agent and a physiological buffering agent for the preferential delivery of said exogenous nucleic acid into smooth muscle cells of coronary arteries over cardiomyocytes.

38. The article of manufacture of claim 37, wherein said packaging material comprises a label or package insert indicating that said exogenous nucleic acid is to be used with cardiac perfusion buffer comprising 0.5 mM Ca²⁺, 100 mM Na¹⁺, said endothelial cell permeability enhancing agent is 1.0×10⁻³ mM histamine and said physiological buffering agent is 20 mM Hepes.

39. An article of manufacture for the efficient delivery of an exogenous nucleic acid into cardiomyocytes, said article of manufacture comprising an exogenous nucleic acid and packaging material, wherein said packaging material comprises a label or package insert indicating that said exogenous nucleic acid is to be used with a cardiac perfusion buffer comprising 0.04-0.06 mM Ca²⁺, 40-60 mM Na¹⁺, an endothelial cell permeability enhancing agent and a physiological buffering agent for the efficient delivery of said exogenous nucleic acid into cardiomyocytes.

40. The article of manufacture of claim 39, wherein said packaging material comprises a label or package insert indicating that said exogenous nucleic acid is to be used with a cardiac perfusion buffer comprising 0.05 mM Ca²⁺, 50 mM Na¹⁺, said endothelial cell permeability enhancing agent is 1.0×10⁻² mM histamine and said physiological buffering agent is 20 mM Hepes.

41. The article of manufacture of claim 37 or 39, wherein said exogenous nucleic acid comprises a viral vector transgene construct.

42. A selectively transfected mammalian heart, wherein said heart comprises an exogenous nucleic acid, wherein said exogenous nucleic acid is present in the medial cells of coronary arteries and is substantially absent from cardiomyocytes.

43. A selectively transfected mammalian heart, wherein said heart comprises a first exogenous nucleic acid, wherein said first exogenous nucleic acid is present in the medial cells of coronary arteries and is substantially absent from cardiomyocytes, and a second exogenous nucleic acid, wherein said second exogenous nucleic acid is present in cardiomyocytes and substantially absent from the medial cells of coronary arteries.

44. The mammalian heart of claim 42 or 43, wherein said mammalian heart is a human heart.

45. The mammalian heart of claim 42 or 43, wherein said mammalian heart is a non-human heart.

46. The mammalian heart of claim 42 or 43, wherein said mammalian heart is a porcine heart.

47. A kit for the preferential delivery of an exogenous nucleic acid into smooth muscle cells of coronary arteries over cardiomyocytes, said kit comprising cardiac perfusion buffer and packaging material, wherein said cardiac perfusion buffer comprises 0.4-0.6 mM Ca²⁺, 80-120 mM Na¹⁺, an endothelial cell permeability enhancing agent and a physiological buffering agent, wherein said packaging material comprises a label or package insert indicating that said cardiac perfusion buffer can be used for the preferential delivery of an exogenous nucleic acid into smooth muscle cells of coronary arteries over cardiomyocytes.

48. The kit of claim 47, wherein said cardiac perfusion buffer comprises 0.5 mM Ca²⁺, 100 mM Na¹⁺, said endothelial cell permeability enhancing agent is 1.0×10⁻³ mM histamine and said physiological buffering agent is 20 mM Hepes.

49. A kit for the efficient delivery of an exogenous nucleic acid into cardiomyocytes, said kit comprising cardiac perfusion buffer and packaging material, wherein said cardiac perfusion buffer comprises 0.04-0.06 mM Ca²⁺, 40-60 mM Na¹⁺, an endothelial cell permeability enhancing agent and a physiological buffering agent, wherein packaging material comprises a label or package insert indicating that said cardiac perfusion buffer can be used for the efficient delivery of an exogenous nucleic acid into cardiomyocytes.

50. The kit of claim 49, wherein said cardiac perfusion buffer comprises 0.05 mM Ca²⁺, 50 mM Na¹⁺, said endothelial cell permeability enhancing agent is 1.0×10⁻² mM histamine and said physiological buffering agent is 20 mM Hepes.

51. The kit claim 47 or 49 further comprising an exogenous nucleic acid.
